(19) United States
(12) Patent Application Publication
(10) Pub. No.: US 2009/0036530 A1
(43) Pub. Date: Feb. 5, 2009

(54) Crystalline acid of lipoxin A4 analogs and method of making

(75) Inventors: Klaus Bartel, Berlin (DE); Danja Grossbach, Berlin (DE); William J. Guilford, Belmont, CA (US); Michael Sander, Frechen (DE); Gabriele Winter, Schonfliess (DE)

Correspondence Address: JACQUELINE S LARSON, 245 AVINGTON ROAD, ALAMEDA, CA, 94502, US

(73) Assignee: Bayer Schering Pharma Aktiengesellschaft, Berlin (DE)

(21) Appl. No.: 12/215,928

(22) Filed: Jun. 30, 2008

Related U.S. Application Data
(63) Continuation-in-part of application No. 11/999,003, filed on Dec. 3, 2007.
(60) Provisional application No. 60/872,824, filed on Dec. 4, 2006.

(51) Int. Cl. A61K 31/192; C07C 59/64; A61P 29/00

(52) U.S. Cl. 514/559; 562/470

(57) ABSTRACT

This invention is directed to a crystalline acid of a lipoxin A₄ analog of Formula (II):

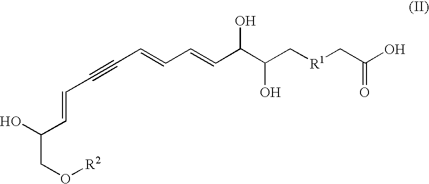

wherein:
R¹ is —O—, —S(O)ₜ— (where t is 0, 1 or 2), or a straight or branched alkylene chain; and
R² is aryl (optionally substituted by one or more substituents selected from alkyl, alkoxy, halo, haloalkyl and haloalkoxy) or aralkyl (optionally substituted by one or more substituents selected from f alkyl, alkoxy, halo, haloalkyl and haloalkoxy);
   and wherein the compound of Formula (II) is a single stereoisomer or any mixture of stereoisomers.

This crystalline acid is useful in treating disease-states characterized by inflammation, such as inflammatory and autoimmune disorders or pulmonary or respiratory tract inflammations in humans. Methods of preparing the crystalline acid are also described.

This application is a continuation-in-part of U.S. application Ser. No. 11/999,003, filed Dec. 3, 2007 and further claims the benefit of Provisional U.S. application Ser. No. 60/872,824, filed Dec. 4, 2006, the entire disclosures of both applications being incorporated herein by reference.

### FIELD OF THE INVENTION

The invention relates to the solid-state crystalline acids of lipoxin A₄ analogs, their use in treating a disease-state characterized by inflammation, and pharmaceutical compositions containing the crystalline acids of the analogs and processes for their preparation.

### BACKGROUND OF THE INVENTION

Lipoxins, together with leukotrienes, prostaglandins, and thromboxanes, constitute a group of biologically active oxygenated fatty acids collectively referred to as the eicosanoids. Eicosanoids are all synthesized de novo from membrane phospholipid via the arachidonic acid cascade of enzymes. Since their initial discovery in 1984, it has become apparent that lipoxins, which are a structurally unique class of eicosanoids, possess potent anti-inflammatory properties that suggest they may have therapeutic potential (Serhan, C. N., Prostaglandins (1997), Vol. 53, pp. 107-137; O'Meara, Y. M. et al., Kidney Int. (Suppl.) (1997), Vol. 58, pp. S56-S61; Brady, H. R. et al., Curr. Opin. Nephrol. Hypertens. (1996), Vol. 5, pp. 20-27; and Serhan, C. N., Biochem. Biophys. Acta. (1994), Vol. 1212, pp. 1-25). Of particular interest is the ability of lipoxins to antagonize the pro-inflammatory functions of leukotrienes in addition to other inflammatory agents such as platelet activating factor, fMLP (formyl-Met-Leu-Phe) peptide, immune complexes, and TNFα. Lipoxins are thus potent anti-neutrophil agents which inhibit polymorphoneutrophil (PMN) chemotaxis, homotypic aggregation, adhesion, migration across endothelial and epithelial cells, margination/diapedesis and tissue infiltration (Lee, T. H., et al., Clin. Sci. (1989), Vol. 77, pp. 195-203; Fiore, S., et al., Biochemistry (1995), Vol. 34, pp. 16678-16686; Papyianni, A., et al., J. Immunol. (1996), Vol. 56, pp. 2264-2272; Hedqvist, P., et al., Acta. Physiol Scand. (1989), Vol. 137, pp. 157-572; Papyianni, A., et al., Kidney Intl. (1995), Vol. 47, pp. 1295-1302). In addition, lipoxins are able to down-regulate endothelial P-selectin expression and adhesiveness for PMNs (Papyianni, A., et al., J. Immunol. (1996), Vol. 56, pp. 2264-2272), bronchial and vascular smooth muscle contraction, mesangial cell contraction and adhesiveness (Dahlen, S. E., et al., Adv. Exp. Med. Biol. (1988), Vo. 229, pp. 107-130; Christie, P. E., et al., Am. Rev. Respir. Dis. (1992), Vol. 145, pp. 1281-1284; Badr, K. F., et al., Proc. Natl. Acad. Sci. (1989), Vol. 86, pp. 3438-3442; and Brady, H. R., et al., Am. J. Physiol. (1990), Vol. 259, pp. F809-F815) and eosinophil chemotaxis and degranulation (Soyombo, O., et al., Allergy (1994), Vol. 49, pp. 230-234).

This unique anti-inflammatory profile of lipoxins, particularly lipoxin A₄, has prompted interest in exploiting their potential as therapeutics for the treatment of inflammatory or autoimmune disorders and pulmonary and respiratory tract inflammation. Such disorders and inflammations that exhibit a pronounced inflammatory infiltrate are of particular interest and include, but are not limited to, inflammatory bowel diseases such as Crohn's disease, dermatologic diseases (such as psoriasis), rheumatoid arthritis, and respiratory disorders (such as asthma).

As with other endogenous eicosanoids, naturally-occurring lipoxins are unstable products that are rapidly metabolized and inactivated (Serhan, C. N., Prostaglandins (1997), Vol. 53, pp. 107-137). This has limited the development of the lipoxin field of research, particularly with respect to in vivo pharmacological assessment of the anti-inflammatory profile of lipoxins. Several U.S. Patents have been issued directed to compounds having the active site of lipoxin A₄, but with a longer tissue half-life. See, e.g., U.S. Pat. Nos. 5,441,951 and 5,648,512. These compounds retain lipoxin A₄ receptor binding activity and the potent in vitro and in vivo anti-inflammatory properties of natural lipoxins (Takano, T., et al., J. Clin. Invest. (1998), Vol. 101, pp. 819-826; Scalia, R., et al., Proc. Natl. Acad. Sci. (1997), Vol. 94, pp. 9967-9972; Takano, T., et al., J. Exp. Med. (1997), Vol. 185, pp. 1693-1704; Maddox, J. F., et al., J. Biol. Chem. (1997), Vol. 272, pp. 6972-6978; Serhan, C. N., et al., Biochemistry (1995), Vol. 34, pp. 14609-14615).

Lipoxin A₄ analogs of interest to the invention are disclosed in U.S. Pat. No. 6,831,186 and in U.S. Patent Application Publication No. 2004/0162433.

It is recognized in the art that it is particularly advantageous that a solid pharmaceutical substance is crystalline, rather than amorphous. Typically during the formation of a crystalline solid by crystallisation from a solution, a purification of the crystalline product is obtained. A crystalline solid state form can be very well characterized and usually shows a higher stability in comparison to an amorphous phase. By using a crystalline solid as drug substance or ingredient of a drug product, a potential recrystallisation of the amorphous phase, including the change of the characteristics of the drug substance or drug product, is avoided. Accordingly, there exists a need for a stable crystalline solid-state form of the lipoxin A₄ analogs disclosed in U.S. Pat. No. 6,831,186 and in U.S. Patent Application Publication No. 2004/0162433.

### SUMMARY OF THE INVENTION

This invention is directed to a potent, selective and metabolically/chemically stable crystalline acid of a lipoxin A₄ analog and its use in treating disease-states characterized by inflammation, such as inflammatory or autoimmune disorders and pulmonary or respiratory tract inflammation in mammals, particularly in humans.

Accordingly, in one aspect this invention is directed to a crystalline free acid of a lipoxin A₄ analog of Formula (II):

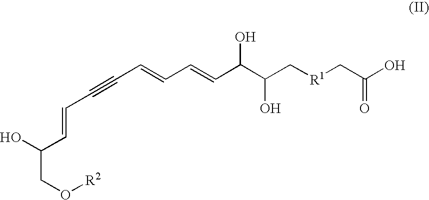

wherein:
R¹ is —O—, —S(O)ₜ— (where t is 0, 1 or 2), or a straight or branched alkylene chain; and
R² is aryl (optionally substituted by one or more substituents selected from alkyl, alkoxy, halo, haloalkyl and haloalkoxy) or aralkyl (optionally substituted by one or more substituents selected from alkyl, alkoxy, halo, haloalkyl and haloalkoxy);
   and wherein the compound of Formula (II) is a single stereoisomer or any mixture of stereoisomers.

The present invention encompasses all of the crystalline forms of the free acid of Formula (II).

In another aspect, this invention is directed to a method of preparing the crystalline form of the acid of Formula (II), the method comprising i) mixing an alkali hydroxide, in a suitable solvent, together with an ester corresponding to the acid of Formula (II), in a suitable solvent; ii) adjusting the pH of the resulting mixture to pH 3-4; iii) after crystals begin to form, further adjusting the pH of the mixture to pH 1-3; iv) isolating the resulting crystals from the resulting suspension; and v) drying the isolated crystals, to give the crystalline acid.

In a further aspect, this invention is directed to pharmaceutical compositions comprising a therapeutically effective amount of a crystalline acid of Formula (II), as set forth above, and a pharmaceutically acceptable excipient or mixture of excipients.

In another aspect, this invention is directed to the use of a crystalline acid of Formula (II), as described above, for the manufacture of a medicament for treating a mammal having a disease-state characterized by inflammation, such as for example an inflammatory or autoimmune disorder or a pulmonary or respiratory tract inflammation.

In another aspect, this invention is directed to methods of treating a disease-state in a mammal, such as a human, characterized by inflammation, wherein the method comprises administering to the mammal in need thereof a therapeutically effective amount of a crystalline acid of Formula (II) as described above. The disease-state may be, for example, an inflammatory or autoimmune disorder or a pulmonary or respiratory tract inflammation.

### BRIEF DESCRIPTION OF THE DRAWINGS

FIG. 1 is the sorption isotherm of polymorph I of crystalline 2-((2S,3R,4E,6E,10E,12S)-13-(4-fluorophenoxy)-2,3,12-trihydroxytrideca-4,6,10-trien-8-ynyloxy)acetic acid, illustrating its hygroscopicity.

FIG. 2 shows the X-ray powder pattern of polymorph I of 2-((2S,3R,4E,6E,10E,12S)-13-(4-fluorophenoxy)-2,3,12-trihydroxytrideca-4,6,10-trien-8-ynyloxy)acetic acid.

FIG. 3 shows the X-ray powder pattern of the hydrate of 2-((2S,3R,4E,6E,10E,12S)-13-(4-fluorophenoxy)-2,3,12-trihydroxytrideca-4,6,10-trien-8-ynyloxy)acetic acid.

### DETAILED DESCRIPTION OF THE INVENTION

All of the references cited herein, including U.S. patents, U.S. published patent applications and journal articles, are incorporated in full by reference herein.

### A. Definitions

As used herein the singular forms “a”, “an”, and “the” include plural referents unless the context clearly dictates otherwise. For example, “a compound” refers to one or more of such compounds, while “the enzyme” includes a particular enzyme as well as other family members and equivalents thereof as known to those skilled in the art.

All percentages herein are by volume, unless otherwise indicated.

Furthermore, as used in the specification and appended claims, unless specified to the contrary, the following terms have the meaning indicated:

“Alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl(isopropyl), n-butyl, n-pentyl, 1,1-dimethylethyl(t-butyl), and the like.

“Alkylene chain” refers to a straight or branched divalent hydrocarbon chain consisting solely of carbon and hydrogen, containing no unsaturation and having from one to eight carbon atoms, e.g., methylene, ethylene, propylene, n-butylene, and the like.

“Alkoxy” refers to a radical of the formula —ORₐ where Rₐ is an alkyl radical as defined above.

“Aryl” refers to a phenyl or naphthyl radical. Unless stated otherwise, the aryl radical may be optionally substituted by one or more substituents selected from the group consisting of alkyl, alkoy, halo, haloalkyl or haloalkoxy. Unless stated otherwise specifically in the specification, it is understood that such substitution can occur on any carbon of the aryl radical.

“Aralkyl” refers to a radical of the formula —RₐR_{b} where Rₐ is an alkyl radical as defined above and R_{b} is an aryl radical as defined above, e.g., benzyl and the like. The aryl radical may be optionally substituted as described above.

“Halo” refers to bromo, chloro, iodo or fluoro.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1-fluoromethyl-2-fluoroethyl (1,3-difluoroisopropyl), 3-bromo-2-fluoropropyl, 1-bromomethyl-2-bromoethyl (1,3-dibromoisopropyl), and the like.

“Haloalkoxy” refers to a radical of the formula —OR_{c} where R_{c} is an haloalkyl radical as defined above, e.g., trifluoromethoxy, difluoromethoxy, trichloromethoxy, 2,2,2-trifluoroethoxy, 1-fluoromethyl-2-fluoroethoxy, 3-bromo-2-fluoropropoxy, 1-bromomethyl-2-bromoethoxy, and the like.

“Clathrates” as used herein refers to substances which fix gases, liquids or compounds as inclusion complexes so that the complex may be handled in solid form and the included constituent (or “guest” molecule) is subsequently released by the action of a solvent or by melting. The term “clathrate” is used interchangeably herein with the phrase “inclusion molecule” or with the phrase “inclusion complex”. Clathrates used in the instant invention are prepared from cyclodextrins. Cyclodextrins are widely known as having the ability to form clathrates (i.e., inclusion compounds) with a variety of molecules. See, for example, Inclusion Compounds, edited by J. L. Atwood, J. E. D. Davies, and D. D. MacNicol, London, Orlando, Academic Press, 1984; Goldberg, I., “The Significance of Molecular Type, Shape and Complementarity in Clathrate Inclusion”, Topics in Current Chemistry (1988), Vol. 149, pp. 2-44; Weber, E. et al., “Functional Group Assisted Clathrate Formation—Scissor-Like and Roof-Shaped Host Molecules”, Topics in Current Chemistry (1988), Vol. 149, pp. 45-135; and MacNicol, D. D. et al., “Clathrates and Molecular Inclusion Phenomena”, Chemical Society Reviews (1978), Vol. 7, No. 1, pp. 65-87. Conversion into cyclodextrin clathrates is known to increase the stability and solubility of certain compounds, thereby facilitating their use as pharmaceutical agents. See, for example, Saenger, W., “Cyclodextrin Inclusion Compounds in Research and Industry”, Angew. Chem. Int. Ed. Engl. (1980), Vol. 19, pp. 344-362; U.S. Pat. No. 4,886,788 (Schering AG); U.S. Pat. No. 6,355,627 (Takasago); U.S. Pat. No. 6,288,119 (Ono Pharmaceuticals); U.S. Pat. No. 6,110,969 (Ono Pharmaceuticals); U.S. Pat. No. 6,235,780 (Ono Pharmaceuticals); U.S. Pat. No. 6,262,293 (Ono Pharmaceuticals); U.S. Pat. No. 6,225,347 (Ono Pharmaceuticals); and U.S. Pat. No. 4,935,446 (Ono Pharmaceuticals).

“Cyclodextrin” refers to cyclic oligosaccharides consisting of at least six glucopyranose units which are joined together by α(1-4) linkages. The oligosaccharide ring forms a torus with the primary hydroxyl groups of the glucose residues lying on the narrow end of the torus. The secondary glucopyranose hydroxyl groups are located on the wider end. Cyclodextrins have been shown to form inclusion complexes with hydrophobic molecules in aqueous solutions by binding the molecules into their cavities. The formation of such complexes protects the “guest” molecule from loss of evaporation, from attack by oxygen, visible and ultraviolet light and from intra- and intermolecular reactions. Such complexes also serve to “fix” a volatile material until the complex encounters a warm moist environment, at which point the complex will dissolve and dissociate into the guest molecule and the cyclodextrin. For purposes of this invention, the six-glucose unit containing cyclodextrin is specified as α-cyclodextrin, while the cyclodextrins with seven and eight glucose residues are designated as β-cyclodextrin and γ-cyclodextrin, respectively. The most common alternative to the cyclodextrin nomenclature is the naming of these compounds as cycloamyloses.

As used herein, compounds which are “commercially available” may be obtained from standard chemical supply houses and other commercial sources including, but not limited to, Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), and Wako Chemicals USA, Inc. (Richmond Va.).

“Mammal” includes humans and domesticated animals, such as cats, dogs, swine, cattle, sheep, goats, horses, rabbits, and the like.

As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogus reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (such as, for example, those listed above) provide custom synthesis services.

“Optional” or “optionally” or “may be” means that the subsequently described event or circumstance may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Polymorphs” refers to polymorphic forms of the acids of the invention. Solids exist in either amorphous or crystalline forms. In the case of crystalline forms, molecules are systematically positioned in 3-dimensional lattice sites. When a compound crystallizes from a solution or slurry, it may crystallize with different spatial lattice arrangements, a property referred to as “polymorphism,” with the different crystal forms individually being referred to as a “polymorph”. Different polymorphic forms of a given substance may differ from each other with respect to one or more physical properties, such as solubility and dissolution, true density, crystal shape, compaction behavior, flow properties, and/or solid state stability. In the case of a chemical substance that exists in two (or more) polymorphic forms, the unstable forms generally convert to the more thermodynamically stable forms at a given temperature after a sufficient period of time. When this transformation is not rapid, the thermodynamically unstable form is referred to as the “metastable” form. However, the metastable form may exhibit sufficient chemical and physical stability under normal storage conditions to permit its use in a commercial form. In this case, the metastable form, although less stable, may exhibit properties desirable over those of the stable form, such as enhanced solubility or better oral bioavailability.

“Solvate” refers to an aggregate that comprises one or more molecules of a compound of the invention with one or more molecules of solvent or a non stoichiometric content of a solvent. The solvent may be water, in which case the solvate is called a hydrate. Alternatively, the solvent may be an organic solvent. Thus, the acids of lipoxin A₄ analogs of Formula (II) may exist as a hydrate, including a monohydrate, dihydrate, hemihydrate, sesquihydrate, trihydrate, tetrahydrate, the dehydrated hydrates with their non-stoichiometric water content, and the like, as well as the corresponding solvated forms. The acids of Formula (II) may be true solvates, while in other cases the salts may merely retain adventitious water or be a mixture of water plus some adventitious solvent. Additionally, the acids of Formula (II) may exist in a crystalline anhydrous form.

See, e.g., Byrn, S et al. “Solid State Chemistry of Drugs”, SSCI (1999), for a discussion of polymorphs and solvates, their characterization and properties, and relevance for drug substances and drug products; and Stahl, P and Wermuth, C “Handbook of Pharmaceutical Salts”, Wiley (2002), for a discussion of salts, their preparation and properties.

As used herein, “suitable conditions” for carrying out a synthetic step are explicitly provided herein or may be discerned by reference to publications directed to methods used in synthetic organic chemistry. The reference books and treatises set forth above that detail the synthesis of reactants useful in the preparation of compounds of the present invention, will also provide suitable conditions for carrying out a synthetic step according to the present invention.

“Suitable solvent” refers to any solvent that is compatible with the components of the reaction and the reaction conditions. The term encompasses one solvent or a mixture of solvents and includes, but is not limited to organic solvents and water. Suitable solvents are known to those of skill in the art or may be discerned by reference to publications directed to methods used in synthetic organic chemistry.

“Therapeutically effective amount” refers to that amount of a acid of the invention which, when administered to a mammal, particularly a human, in need thereof, is sufficient to effect treatment, as defined below, for a disease-state characterized by inflammation. The amount of a acid of the invention which constitutes a “therapeutically effective amount” will vary depending on the salt, its solvated form, the disease-state to be treated and its severity, the age of the mammal to be treated, and the like, but can be determined routinely by one of ordinary skill in the art.

“Treating” or “treatment” as used herein covers the treatment of a disease-state characterized by inflammation in a mammal, preferably a human, such as for example an inflammatory or autoimmune disorder or a pulmonary or respiratory tract inflammation, and includes:

(i) preventing the disorder or inflammation from occurring in a mammal, in particular, when such mammal is predisposed to the disorder but has not yet been diagnosed as having it;

(ii) inhibiting the disorder or inflammation, i.e., arresting its development; or

(iii) relieving the disorder or inflammation, i.e., causing regression of the disorder or inflammation.

The acids of Formula (II) may contain one or more asymmetric centers and may thus give rise to enantiomers, diastereomers, and other stereoisomeric forms that may be defined, in terms of absolute stereochemistry, as (R)− or (S)− or as (D)− or (L)−. The present invention is meant to include all such possible isomers, as well as their racemic and optically pure forms. Optically active (+) and (−), (R)− and (S)−, or (D)− and (L)− isomers may be prepared using chiral synthons or chiral reagents, or resolved using conventional techniques, such as HPLC using a chiral column. When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

The nomenclature used herein is a modified form of the I.U.P.A.C. nomenclature system. For example, the acid of Formula (II) wherein R¹ is —O— and R² is 4-fluorophenyl, i.e., the acid having the following formula:

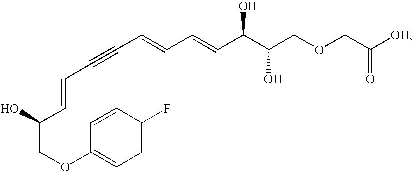

is named herein as 2-((2S,3R,4E,6E,10E,12S)-13-(4-fluorophenoxy)-2,3,12-trihydroxytrideca-4,6,10-trien-8-ynyloxy)acetic acid.

### B. Crystalline Acids of the Invention

Compounds of Formula (II) are described in detail in U.S. Pat. No. 6,831,186 and in U.S. Patent Application Publication No. 2004/0162433, the pertinent disclosures of which are incorporated in full herein by reference.

However, surprisingly it was found that during earlier development, synthesis of a crystalline form of the acid of Formula (II) was elusive. The crystalline form was not reliably obtained following the procedures described in the above two publications.

It is common knowledge that the crystalline solid-state forms of pharmaceutical acids can dramatically increase the stability of a pharmaceutical agent. It is also known that crystalline forms are more stable than amorphous forms.

Accordingly, investigations were conducted with the goal of finding a synthetic route for preparing a suitable stable solid-state crystalline form of a compound of Formula (II):

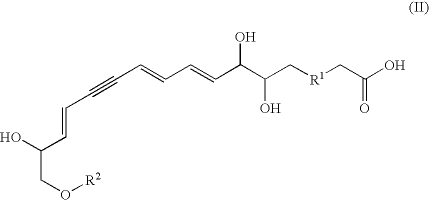

wherein:
R¹ is —O—, —S(O)ₜ— (where t is 0, 1 or 2) or a straight or branched alkylene chain; and
R² is aryl (optionally substituted by one or more substituents selected from alkyl, alkoxy, halo, haloalkyl and haloalkoxy) or aralkyl (optionally substituted by one or more substituents selected from alkyl, alkoxy, halo, haloalkyl and haloalkoxy);
   and wherein the compound of Formula (II) is a single stereoisomer or any mixtures of stereoisomers.

In one embodiment, the invention is directed to a crystalline acid of Formula (II) where R¹ is —O— and R² is phenyl optionally substituted by one or more substituents selected from fluoro, chloro and iodo. In another embodiment, the compound of the invention is a crystalline acid of Formula (II) where R¹ is —O— and R² is 4-fluorophenyl. In a further embodiment, the compound is selected from the group consisting of the following:
2-((2S,3R,4E,6E,10E,12S)-13-(4-fluorophenoxy)-2,3,12-trihydroxytrideca-4,6,10-trien-8-ynyloxy)acetic acid;
2-((2R,3R,4E,6E,10E,12S)-13-(4-fluorophenoxy)-2,3,12-trihydroxytrideca-4,6,10-trien-8-ynyloxy)acetic acid;
2-((2S,3S,4E,6E,10E,12S)-13-(4-fluorophenoxy)-2,3,12-trihydroxytrideca-4,6,10-trien-8-ynyloxy)acetic acid;
2-((2R,3S,4E,6E,10E,12S)-13-(4-fluorophenoxy)-2,3,12-trihydroxytrideca-4,6,10-trien-8-ynyloxy)acetic acid;
2-((2S,3R,4E,6E,10E,12R)-13-(4-fluorophenoxy)-2,3,12-trihydroxytrideca-4,6,10-trien-8-ynyloxy)acetic acid;
2-((2R,3R,4E,6E,10E,12R)-13-(4-fluorophenoxy)-2,3,12-trihydroxytrideca-4,6,10-trien-8-ynyloxy)acetic acid;
2-((2S,3S,4E,6E,10E,12R)-13-(4-fluorophenoxy)-2,3,12-trihydroxytrideca-4,6,10-trien-8-ynyloxy)acetic acid; and
2-((2R,3S,4E,6E,10E,12R)-13-(4-fluorophenoxy)-2,3,12-trihydroxytrideca-4,6,10-trien-8-ynyloxy)acetic acid.
   In yet another embodiment, the invention is directed to crystalline 2-((2S,3R,4E,6E,10E,12S)-13-(4-fluorophenoxy)-2,3,12-trihydroxytrideca-4,6,10-trien-8-ynyloxy)acetic acid:

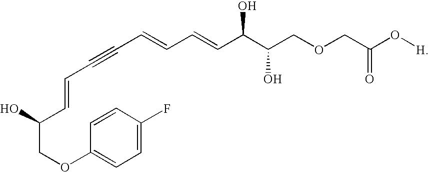

### C. Preparation of Crystalline 2-((2S,3R,4E,6E,10E,12S)-13-(4-fluorophenoxy)-2,3,12-trihydroxytrideca-4,6,10-trien-8-ynyloxy)acetic acid

The methods of preparing the crystalline forms of 2-((2S,3R,4E,6E,10E,12S)-13-(4-fluorophenoxy)-2,3,12-trihydroxytrideca-4,6,10-trien-8-ynyloxy)acetic acid also may be used to prepare crystalline acids of other compounds of Formula (II). In general, the method of preparing the crystalline 2-((2S,3R,4E,6E,10E,12S)-13-(4-fluorophenoxy)-2,3,12-trihydroxytrideca-4,6,10-trien-8-ynyloxy)acetic acid is based on the saponification of an ester having the following Formula (III):

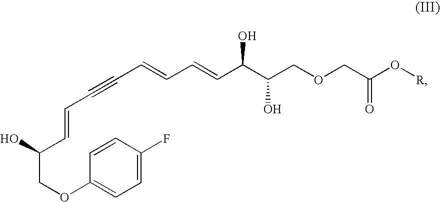

where R is alkyl or aryl, by treatment with a base, followed by acidification of the resulting mixture by treatment with an acid. The resulting mixture forms a suspension, which is then optionally cooled. The crystals are isolated from suspension and dried to yield the desired hydrate form of the crystalline acid.

In a particular embodiment, the ester of Formula (III) is dissolved in a suitable solvent, such as methanol, ethanol or tetrahydrofuran (THF), for example. Then an alkali hydroxide base, such as sodium hydroxide or potassium hydroxide for example, is dissolved in a suitable solvent, such as methanol, ethanol or water, for example, or mixtures of these solvents. The solution of the alkali hydroxide is added to the solution of the ester or vice versa. Additional water is added, if necessary, in an amount sufficient to effect suitable crystallization of the product upon acidification.

The resulting mixture is acidified with a suitable acid, such as hydrochloric acid (HCl) to form a suspension that is isolated and dried. The drying procedure of the isolated crystals defines the “hydrate form” of the crystalline acid prepared.

### D. Solid State Forms of 2-((2S,3R,4E,6E,10E,12S)-13-(4-fluorophenoxy)-2,3,12-trihydroxytrideca-4,6,10-trien-8-ynyloxy)acetic acid

2-((2S,3R,4E,6E,10E,12S)-13-(4-fluorophenoxy)-2,3,12-trihydroxytrideca-4,6,10-trien-8-ynyloxy)acetic acid, prepared according to the present invention, exists as a crystalline anhydrous form, named polymorph 1, and as a crystalline hydrate form, as well as an amorphous phase.

TABLE 1
X-ray powder diffraction data of polymorph I and the hydrate -
d-values (germanium-monochromatized CuKα₁-radiation)

| polymorph I | hydrate |
|---|---|
| d (Å) | d (Å) |
| 20.48 | 9.8 |
| 10.05 | 4.6 |
| 4.46 | 4.4 |
| 4.41 | 4.3 |
| 4.34 | 4.0 |
| 4.17 | 3.5 |
| 4.03 | 3.4 |
| 3.67 | |
| 3.51 | |
| 3.35 | |
| 3.14 | |

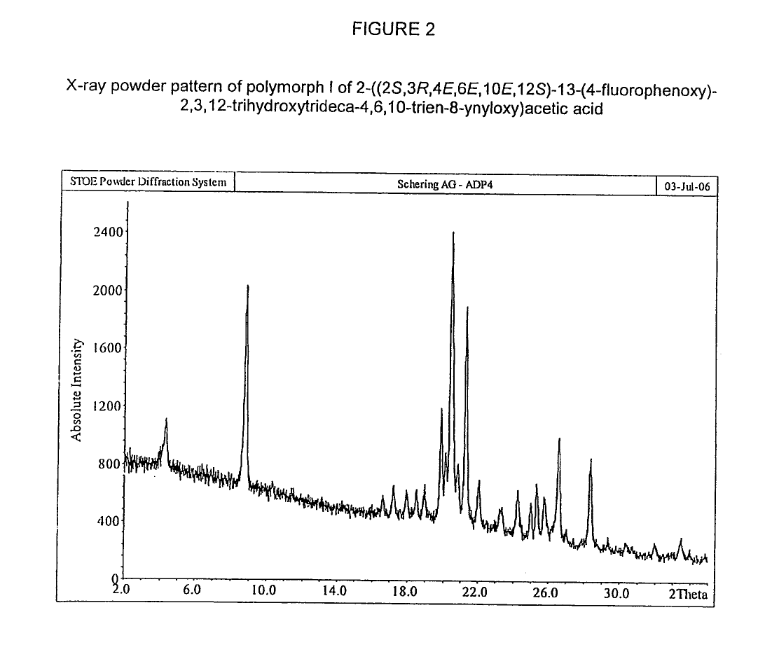

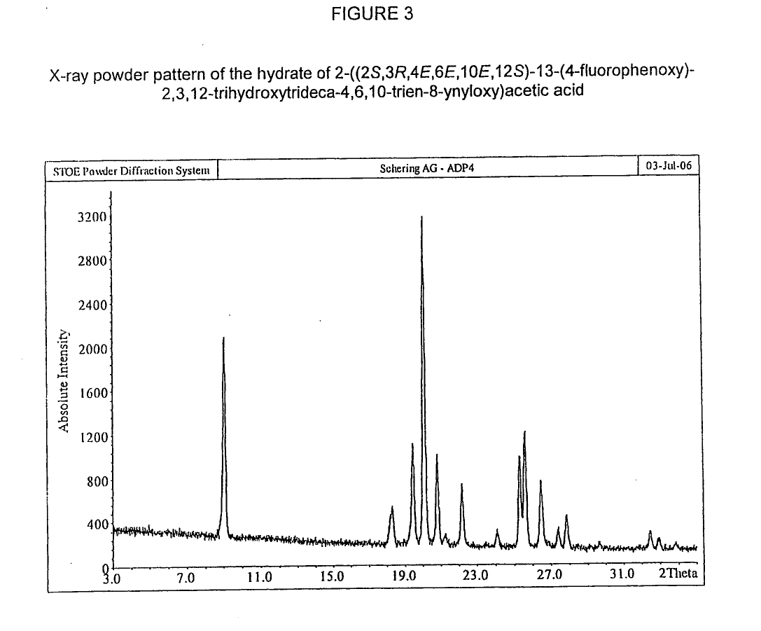

The anhydrous form (polymorph 1) of the crystalline 2-((2S,3R,4E,6E,10E,12S)-13-(4-fluorophenoxy)-2,3,12-trihydroxytrideca-4,6,10-trien-8-ynyloxy)acetic acid exhibits characteristic peaks at d=20.48 Å and at d=4.34, while the hydrate form of the crystalline acid of 2-((2S,3R,4E,6E,10E,12S)-13-(4-fluorophenoxy)-2,3,12-trihydroxytrideca-4,6,10-trien-8-ynyloxy)acetic acid exhibits characteristic peaks at d=9.8 Å and at d=4.6 Å (see, FIGS. 2 and 3).

Data collection for the XRPD was carried out in transmission mode on automated STOE Powder Diffractometers using germanium-monochromatized CuKα₁-radiation (λ=1.5406 Å). The X-ray tube with copper anode was operated by 40 kV and 30 mA. The 2Θ scans were performed using the small linear position sensitive detector with an angular resolution of 0.08° between 3°≦2Θ≦35° and 2°≦2Θ≦35° (stepwidth 0.5°) or 7°≦2Θ≦35° (stepwidth 0.5°) if the wellplate was used. The samples were enclosed between two polyacetate films held together by double-sided adhesive tape or between two aluminum foils to avoid the influence of the humidity during measurement. Data acquisition and evaluation were performed using the STOE WinX^{pow} software package. One of ordinary skill in the art will appreciate that an X-ray diffraction pattern may be obtained with a measurement error that is dependent upon the measurement conditions employed. In particular, it is generally known that intensities in an X-ray diffraction pattern may fluctuate depending upon crystal habitus of the material and measurement conditions employed. It is further understood that relative intensities may also vary depending upon experimental conditions and, accordingly, the exact order of intensity should not be taken into account. Additionally, a measurement error of diffraction angle Theta for a conventional X-ray diffraction pattern at a given temperature is typically about ±0.1, and such degree of measurement error should be taken into account as pertaining to the aforementioned diffraction angles. Consequently, the term “about” when used herein in reference to X-ray powder diffraction patterns means that the crystal forms of the instant invention are not limited to the crystal forms that provide X-ray diffraction patterns completely identical to the X-ray diffraction patterns depicted in the accompanying Figure disclosed herein. Any crystal form that provides X-ray diffraction patterns that is substantially identical to those disclosed in the accompanying FIGURES falls within the scope of the present invention. The ability to ascertain whether the polymorphic forms of a compound are the same albeit the X-ray diffraction patterns are not completely identical is within the purview of one of ordinary skill in the art.

### E. Hygroscopicity of Crystalline 2-((2S,3R,4E,6E,10E,12S)-13-(4-fluorophenoxy)-2,3,12-trihydroxytrideca-4,6,10-trien-8-ynyloxy)acetic acid

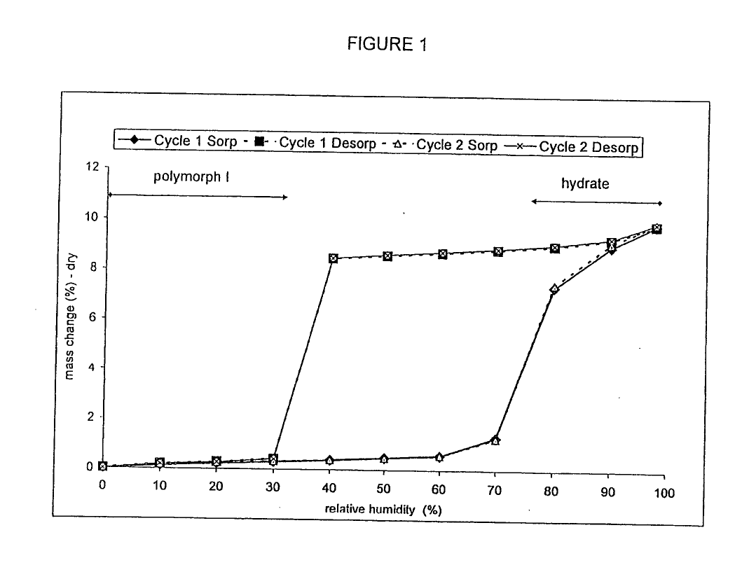

Polymorph I of the free acid is transformed above 60% relative humidity into the hydrate. It is hygroscopic and adsorbs at 80% relative humidity approx. 8% moisture, which corresponds to a dihydrate of the substance. The hydration is reversible. Below 40% relative humidity a transformation into polymorph I is observed. The sorption isotherm is given in FIG. 1.

Data collection for the hygroscopicity studies was carried out in an automated water sorption analyzer. Approximately 10 mg of the investigated solid state form of the crystalline acid was exposed to a continuous flow of nitrogen with predetermined and constant relative humidity. The rate of sweep gas was set at 200 cm³/min. For a basic assay, two full cycles (sorption/desorption) were measured at 25° C. The measurement was started at 0% relative humidity in order to remove surface water. Once the constant mass was achieved, the next humidity was automatically set. The water sorption/desorption was investigated in steps of 10% between 0% and 90% relative humidity under the criteria for initiation of the first step as set forth herein. Additionally, the sorption at about 98% relative humidity was investigated. Data was acquired using the DVSWin software.

### F. Relative Chemical Stability of 2-((2S,3R,4E,6E,10E,12S)-13-(4-fluorophenoxy)-2,3,12-trihydroxytrideca-4,6,10-trien-8-ynyloxy)acetic acid

In addition to the investigation of the stability domains—as a function of relative humidity and temperature—of polymorph I of the acid, the relative chemical stabilities have been determined.

The relative chemical stability of polymorph I of 2-((2S,3R,4E,6E,10E,12S)-13-(4-fluorophenoxy)-2,3,12-trihydroxytrideca-4,6,10-trien-8-ynyloxy)acetic acid over a period of 4 weeks at different storage conditions is shown in the following Table 2.

TABLE 2

| | 25° C., dry | 25° C./60% RH | 40° C., dry | 40° C./75% RH | weeks |
|---|---|---|---|---|---|
| HPLC- | 97, 36 | 97, 36 | 97, 36 | 97, 36 | 0 |
| purity [%] | 97, 49 | 97, 49 | 96, 92 | 95, 88 | 2 |
| | 97, 48 | 97, 72 | 96, 41 | 92, 51 | 4 |

### I. Utility of the Crystalline Acids of the Invention

The crystalline acids of Formula (II) have biological activity similar to that of natural lipoxin A₄, but with an enhanced resistance to chemical and metabolic degradation. Accordingly, the crystalline acids of Formula (II) are useful in treating inflammatory or autoimmune disorders in mammals, such as, e.g., in humans. In particular, a crystalline acid of Formula (II) is useful in inhibiting acute or chronic inflammation or an inflammatory or autoimmune response that is mediated by neutrophils, eosinophils, T lymphocytes, NK cells or other immune cells that contribute to the pathogenesis of inflammatory, immune or autoimmune diseases. The crystalline acids of Formula (II) are also useful in the treatment of proliferative disorders including, but not limited to, those associated with derangements in the inflammatory or immune response, such as cancer. The crystalline acids of Formula (II) are also useful as an inhibitor of angiogenic responses in the pathogenesis of cancer.

Accordingly, a crystalline acid of Formula (II) can be used to treat the following inflammatory or autoimmune disorders in mammals, particularly in humans: anaphylactic reactions, allergic reactions, allergic contact dermatitis, allergic rhinitis, chemical and non-specific irritant contact dermatitis, urticaria, atopic dermatitis, psoriasis, fistulas associated with Crohn's disease, pouchitis, septic or endotoxic shock, hemorrhagic shock, shock-like syndromes, capillary leak syndromes induced by immunotherapy of cancer, acute respiratory distress syndrome, traumatic shock, immune- and pathogen-induced pneumonias, immune complex-mediated pulmonary injury and chronic obstructive pulmonary disease, inflammatory bowel diseases (including ulcerative colitis, Crohn's disease and post-surgical trauma), gastrointestinal ulcers, diseases associated with ischemia-reperfusion injury (including acute myocardial ischemia and infarction, acute renal failure, ischemic bowel disease and acute hemorrhagic or ischemic stroke), immune-complex-mediated glomerulonephritis, autoimmune diseases (including insulin-dependent diabetes mellitus, multiple sclerosis, rheumatoid arthritis, osteoarthritis and systemic lupus erythematosus), acute and chronic organ transplant rejection, transplant arteriosclerosis and fibrosis, cardiovascular disorders (including hypertension, atherosclerosis, aneurysm, critical leg ischemia, peripheral arterial occlusive disease and Reynaud's syndrome), complications of diabetes (including diabetic nephropathy, neuropathy and retinopathy), ocular disorders (including macular degeneration and glaucoma), neurodegenerative disorders (including delayed neurodegeneration in stroke, Alzheimer's disease, Parkinson's disease, encephalitis and HIV dementia), inflammatory and neuropathic pain including arthritic pain, periodontal disease including gingivitis, ear infections, migraine, benign prostatic hyperplasia, cancers including, but not limited to, leukemias and lymphomas, prostate cancer, breast cancer, lung cancer, malignant melanoma, renal carcinoma, head and neck tumors and colorectal cancer.

The crystalline acids of Formula (II) are also useful in treating folliculitis induced by inhibitors of epidermal growth factor (EGF) or epidermal growth factor receptor (EGFR) kinase used in the treatment of solid tumors. Clinical trials have revealed folliculitis (inflammation of the hair follicle manifested by severe acne-like skin rash on the face, chest and upper back) as a major dose-limiting side effect of such treatments. Such folliculitis is associated with an infiltration of neutrophils, suggesting products secreted by activated neutrophils to be the cause of the inflammation. The crystalline acids of Formula (II) inhibit neutrophil- or eosinophil-mediated inflammation, and are therefore useful in treating such folliculitis, thereby improving the quality of life of the treated cancer patients but also allowing for the increase of the dosage of the EGF inhibitor or EGFR kinase inhibitor or the extension of the duration of the treatment, resulting in improved efficacy of the desired inhibitor.

The crystalline acids of Formula (II) are also useful in the treatment of pulmonary and respiratory inflammation, including, but not limited to, asthma, chronic bronchitis, bronchiolitis, bronchiolitis obliterans (including such with organizing pneumonia), allergic inflammation of the respiratory tract (including rhinitis and sinusitis), eosinophilic granuloma, pneumonias, pulmonary fibroses, pulmonary manifestations of connective tissue diseases, acute or chronic lung injury, chronic obstructive pulmonary diseases, adult respiratory distress syndrome, and other non-infectious inflammatory disorders of the lung characterized by eosinophil infiltration. For example, a crystalline acid of Formula (II) is useful in the inhibition of: eosinophil-mediated inflammation of the lung or tissues; neutrophil-mediated inflammation of the lung; lymphocyte-mediated inflammation of the lung; cytokine and chemokine production, including interleukin-5, interleukin-13 and eotaxin; lipid mediator generation, including prostaglandin E₂ and cysteinyl leukotrienes; airway hyper-responsiveness; and airway and vascular inflammation.

### J. Testing of the Crystalline Acids of Formula (II)

A hallmark of inflammation is the adhesion and transmigration across endothelium of neutrophils, eosinophils and other inflammatory cells. A similar process is observed for the migration of cells across polarized epithelial cells that occur in the lung, gastrointestinal tract and other organs. Cell culture models of these processes are available and have been used to show that lipoxin A₄ and stable lipoxin A₄ analogs inhibit the transmigration of human neutrophils across human endothelial cells and epithelial cells, including the human intestinal epithelial cell line T₈₄. Accordingly, one of ordinary skill in the art can test a crystalline acid of Formula (II) for its ability to inhibit the transmigration of human neutrophils and eosinophils across human endothelial cells and epithelial cells by performing assays similar to those described in Colgan, S. P., et al., J. Clin. Invest. (1993), Vol. 92, No. 1, pp. 75-82; and Serhan, C. N., et al., Biochemistry (1995), Vol. 34, No. 44, pp. 14609-14615.

The air pouch model and/or the mouse zymosan-induced peritonitis model may be used to evaluate the in vivo efficacy of a crystalline acid of Formula (II) in treating an inflammatory response. These are acute experimental models of inflammation characterized by infiltration of inflammatory cells into a localized area. See, e.g., the in vivo assays described in Ajuebor, M. N., et al., Immunology (1998), Vol. 95, pp. 625-630; Gronert, K., et al., Am. J. Pathol. (2001), Vol. 158, pp. 3-9; Pouliot, M., et al., Biochemistry (2000), Vol. 39. pp. 4761-4768; Clish, C. B., et al., Proc. Natl. Acad. Sci. U.S.A. (1999), Vol. 96, pp. 8247-8252; and Hachicha, M., et al., J. Exp. Med. (1999), Vol. 189, pp. 1923-30.

Animal models (i.e., in vivo assays) may also be utilized to determine the efficacy of the crystalline acids of Formula (II) in treating asthma and related disorders of the pulmonary and respiratory tract. See, e.g., the assays described in De Sanctis, G. T. et al., Journal of Clinical Investigation (1999), Vol. 103, pp. 507-515; and Campbell, E. M., et al., J. Immunol. (1998), Vol. 161, No. 12, pp. 7047-7053.

Alternatively, a crystalline acid of Formula (II) may be tested for its efficacy in the claimed methods of use by employing the assays described in U.S. Pat. No. 6,831,186 and in U.S. Patent Application Publication No. 2004/0162433, the pertinent disclosures of which are incorporated in full in their entireties herein.

### K. Administration of the Crystalline Acids of Formula (II)

Administration of a crystalline acid of Formula (II), as a single stereoisomer or any mixture of stereoisomers, or as a cyclodextrin clathrate thereof, or as a solvate or polymorph, in pure form or in an appropriate pharmaceutical composition, can be carried out via any of the accepted modes of administration or agents for serving similar utilities. Thus, administration can be, for example, orally, nasally, parenterally, pulmonary, topically, transdermally, or rectally, in the form of solid, semi-solid, lyophilized powder, or liquid dosage forms, such as for example, tablets, suppositories, pills, soft elastic and hard gelatin capsules, powders, solutions, suspensions, aerosols, patches, or the like, preferably in unit dosage forms suitable for simple administration of precise dosages. The compositions will include a crystalline acid of the invention as the/an active agent and a conventional pharmaceutical carrier or excipient and, in addition, may include other medicinal agents, pharmaceutical agents, carriers, adjuvants, etc., as are generally known in the art.

Actual methods of preparing such dosage forms are known, or will be apparent, to those skilled in this art; for example, see Remington's Pharmaceutical Sciences, 18th Ed. (Mack Publishing Company, Easton, Pa., 1990). The composition to be administered will, in any event, contain a therapeutically effective amount of a crystalline acid of Formula (II) for treatment of a disease-state characterized by inflammation in accordance with the teachings of this invention.

Generally, depending on the intended mode of administration, the pharmaceutically acceptable compositions will contain about 0.1% to about 99.9% by weight of a crystalline acid of Formula (II) and about 99.9% to about 0.1% by weight of a suitable pharmaceutical excipient.

In one embodiment, the preferred route of administration is oral, using a convenient daily dosage regimen that can be adjusted according to the degree of severity of the disease-state to be treated. For such oral administration, a pharmaceutically acceptable composition containing a crystalline acid of Formula (II) is formed by the incorporation of one or more of the normally employed pharmaceutically acceptable excipient(s). Such compositions take the form of solutions, suspensions, tablets, pills, capsules, powders, sustained release formulations and the like.

Preferably, such compositions will take the form of a capsule, caplet or tablet and therefore will also generally contain a diluent, a disintegrant, a lubricant, and a binder.

A crystalline acid of Formula (II) may also be formulated into a suppository comprising the active ingredient disposed in a carrier that slowly dissolves within the body, such as those normally employed in this capacity.

Liquid pharmaceutically administrable compositions can, for example, be prepared by dissolving, dispersing, etc., a acid of the invention and optional pharmaceutically acceptable adjuvants in a carrier, such as, for example, water, saline, aqueous dextrose, glycerol, ethanol and the like, to thereby form a solution or suspension.

If desired, a pharmaceutical composition of the invention may also contain minor amounts of auxiliary substances such as wetting or emulsifying agents, pH buffering agents, antioxidants, and the like.

A crystalline acid of Formula (II) is administered in a therapeutically effective amount, which will vary depending upon a variety of factors including the activity of the specific compound employed; the metabolic stability and length of action of the crystalline acid of Formula (II); the age, body weight, general health, sex, and diet of the patient; the mode and time of administration; the rate of excretion; the drug combination; the severity of the particular disease-state(s) to be treated; and the host undergoing therapy.

### EXAMPLES

The following Examples further describe the preparation of crystalline 2-((2S,3R,4E,6E,10E,12S)-13-(4-fluorophenoxy)-2,3,12-trihydroxytrideca-4,6,10-trien-8-ynyloxy)acetic acid. The crystalline acids of other lipoxin A analogs of Formula (II) may also be prepared analogously, following the general procedures described herein and exemplified in Examples 1-6.

### Example 1

### Preparation of the anhydrous form of crystalline 2-((2S,3R,4E,6E,10E,12S)-13-(4-fluorophenoxy)-2,3,12-trihydroxytrideca-4,6,10-trien-8-ynyloxy)acetic acid

A solution of 18.3 g of tert-butyl 2-((2S,3R,4E,6E,10E,12S)-13-(4-fluorophenoxy)-2,3,12-trihydroxytrideca-4,6,10-trien-8-ynyloxy)acetate (prepared following the general procedures described in U.S. Pat. No. 6,831,186) in 200 mL of methanol was treated with 20 mL of 1 N hydrogen chloride solution and stirred overnight. The reaction was then cooled in an ice bath and treated with conc. aqueous sodium hydroxide to pH of over 12. After 5 hr, the pH had dropped to about 9 and additional conc. aq. NaOH was added. The reaction was diluted with water and treated with HP-20; the filtrate was concentrated, treated with HP-20 and acidified to a pH of less than 3. The resin was isolated and washed with water. The combined aqueous acid filtrate was set aside at room temperature for several hours. After standing, the filtrate contained solid material, which was isolated, analyzed and determined to be crystalline acid product. The crystals were dried and saved for use as seed crystals.

### Example 2

### Preparation of the anhydrous form of crystalline 2-((2S,3R,4E,6E,10E,12S)-13-(4-fluorophenoxy)-2,3,12-trihydroxytrideca-4,6,10-trien-8-ynyloxy)acetic acid

12.2 Grams of tert-butyl 2-((2S,3R,4E,6E,10E,12S)-13-(4-fluorophenoxy)-2,3,12-trihydroxytrideca-4,6,10-trien-8-ynyloxy)acetate (prepared following the general procedures described in U.S. Pat. No. 6,831,186) were dissolved in 37 mL of methanol, and 6.6 mL of 25% (w/w) aqueous sodium hydroxide solution were added to the ester solution. After 1 h at room temperature, 363 mL of water were added. The pH of the resulting solution was adjusted to pH 4 with 20 mL of 2N HCl. One mg of seed crystals of the desired acid was added and the product began to crystallize. The seed crystals were necessary in this case because of lower purity of the starting ester. An additional 6.7 mL of 2N HCl were added slowly until the mixture was pH 2.5. The resulting suspension was stirred for 2 h at room temperature and then filtered. The resulting crystals were washed with water and dried at 25° C./200 mbar in a vacuum-drying cabinet using nitrogen as a carrier gas. 9.5 Grams of light brown crystals of 2-((2S,3R,4E,6E,10E,12S)-13-(4-fluorophenoxy)-2,3,12-trihydroxytrideca-4,6,10-trien-8-ynyloxy)acetic acid were isolated.

### Example 3

### Preparation of the anhydrous form of crystalline 2-((2S,3R,4E,6E,10E,12S)-13-(4-fluorophenoxy)-2,3,12-trihydroxytrideca-4,6,10-trien-8-ynyloxy)acetic acid

Tert-butyl 2-{[(4E,6E,10E)-(2S,3R,12S)-13-(4-fluorophenoxy)-2,3,12-trihydroxytrideca-4,6,10-trien-8-yn-1-yl]oxy}acetate is dissolved in a toluene/methanol mixture and filtered over a plug of silica gel using mixtures of hexane and tert-butyl methyl ether as eluent. Fractions are collected and concentrated in vacuum.

3.3 Grams of filtered tert-butyl 2-{[(4E,6E,10E)-(2S,3R,12S)-13-(4-fluorophenoxy)-2,3,12-trihydroxytrideca-4,6,10-trien-8-yn-1-yl]oxy}acetate are dissolved in 10 mL of methanol, and 0.89 mL of 25% (w/w) aqueous sodium hydroxide solution are then added. After 1 hr at room temperature, an additional 0.27 mL (w/w) aqueous sodium hydroxide solution are added. After complete conversion. 100 mL of water are added. The pH of the resulting solution is adjusted to pH 4 with 3.7 mL of 2 N hydrochloric acid, and the product starts to crystallize. Additional 1.6 mL of 2 N hydrochloric acid are added slowly until pH 2.5. The suspension is stirred for 1 hr at room temperature. The reaction flask is placed in an ice/water cooling bath and stirred for another hour. Additional 1 mL of hydrochloric acid is added. The suspension is filtered, and the resulting crystals are washed with water and dried at 30° C./200 mbar in a vacuum-drying cabinet using nitrogen as carrier gas. 2.3 Grams of light brown crystals are isolated.

### Example 4

### Preparation of the anhydrate form of the crystalline 2-((2S,3R,4E,6E,10E,12S)-13-(4-fluorophenoxy)-2,3,12-trihydroxytrideca-4,6,10-trien-8-ynyloxy)acetic acid

The anhydrous crystalline acid (15 mg), from Example 2 or 3, e.g., was dissolved at approx. 50° C. in 0.1 mL acetonitrile. The solution was concentrated by slow evaporation of the solvent at room temperature.

### Example 5

### Preparation of the hydrate form of the crystalline 2-((2S,3R,4E,6E,10E,12S)-13-(4-fluorophenoxy)-2,3,12-trihydroxytrideca-4,6,10-trien-8-ynyloxy)acetic acid

The anhydrous crystalline acid (15 mg), from Example 2 or 3, e.g., was dissolved at approx. 100° C. in 0.1 mL water. The obtained solution was concentrated by slow evaporation of the solvent at room temperature.

### Example 6

### Preparation of the hydrate form of the crystalline 2-((2S,3R,4E,6E,10E,12S)-13-(4-fluorophenoxy)-2,3,12-trihydroxytrideca-4,6,10-trien-8-ynyloxy)acetic acid

8 Milligrams of polymorph I of the free acid were stored at 25° C. and 90% relative humidity.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A crystalline acid of Formula (II): 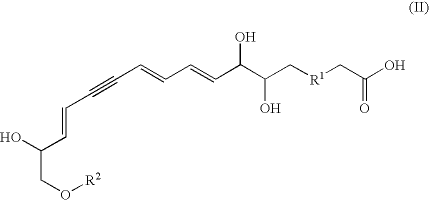
wherein:
R¹ is —O—, —S(O)ₜ— (where t is 0, 1 or 2), or a straight or branched alkylene chain; and
R² is aryl (optionally substituted by one or more substituents selected from alkyl, alkoxy, halo, haloalkyl and haloalkoxy) or aralkyl (optionally substituted by one or more substituents selected from alkyl, alkoxy, halo, haloalkyl and haloalkoxy);
and wherein the compound of Formula (II) is a single stereoisomer or any mixture of stereoisomers.

2. The crystalline acid according to claim 1 wherein R¹ is —O— and R² is phenyl optionally substituted by one or more substituents selected from fluoro, chloro and iodo.

3. The crystalline acid according to claim 2 wherein R¹ is —O— and R² is 4-fluorophenyl, as a single stereoisomer or any mixture of stereoisomers.

4. The crystalline acid according to claim 3, wherein the crystalline acid is:
2-((2S,3R,4E,6E,10E,12S)-13-(4-fluorophenoxy)-2,3,12-trihydroxytrideca-4,6,10-trien-8-ynyloxy)acetic acid;
2-((2R,3R,4E,6E,10E,12S)-13-(4-fluorophenoxy)-2,3,12-trihydroxytrideca-4,6,10-trien-8-ynyloxy)acetic acid;
2-((2S,3S,4E,6E,10E,12S)-13-(4-fluorophenoxy)-2,3,12-trihydroxytrideca-4,6,10-trien-8-ynyloxy)acetic acid;
2-((2R,3S,4E,6E,10E,12S)-13-(4-fluorophenoxy)-2,3,12-trihydroxytrideca-4,6,10-trien-8-ynyloxy)acetic acid;
2-((2S,3R,4E,6E,10E,12R)-13-(4-fluorophenoxy)-2,3,12-trihydroxytrideca-4,6,10-trien-8-ynyloxy)acetic acid;
2-((2R,3R,4E,6E,10E,12R)-13-(4-fluorophenoxy)-2,3,12-trihydroxytrideca-4,6,10-trien-8-ynyloxy)acetic acid;
2-((2S,3S,4E,6E,10E,12R)-13-(4-fluorophenoxy)-2,3,12-trihydroxytrideca-4,6,10-trien-8-ynyloxy)acetic acid; or
2-((2R,3S,4E,6E,10E,12R)-13-(4-fluorophenoxy)-2,3,12-trihydroxytrideca-4,6,10-trien-8-ynyloxy)acetic acid.

5. The crystalline acid according to claim 3, wherein the crystalline acid is 2-((2S,3R,4E,6E,10E,12S)-13-(4-fluorophenoxy)-2,3,12-trihydroxytrideca-4,6,10-trien-8-ynyloxy)acetic acid:

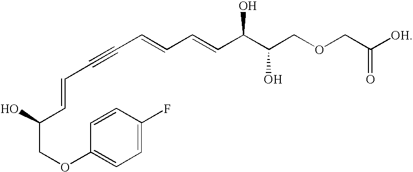

6. The crystalline acid according to claim 1 wherein the crystalline acid is in anhydrous form.

7. The crystalline acid according to claim 1 wherein the crystalline acid is in the form of a hydrate

8. The crystalline acid according to claim 1 wherein the crystalline acid is in the form of a mixture of anhydrate and hydrate.

9. The crystalline acid according to claim 5, wherein the crystalline acid is 2-((2S,3R,4E,6E,10E,12S)-13-(4-fluorophenoxy)-2,3,12-trihydroxytrideca-4,6,10-trien-8-ynyloxy)acetic acid 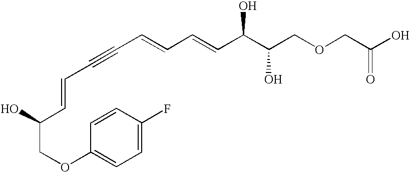
in an anhydrous form which exhibits characteristic peaks at d=20.48 Å and at d=4.34.

10. The crystalline acid according to claim 5, wherein the crystalline acid is 2-((2S,3R,4E,6E,10E,12S)-13-(4-fluorophenoxy)-2,3,12-trihydroxytrideca-4,6,10-trien-8-ynyloxy)acetic acid 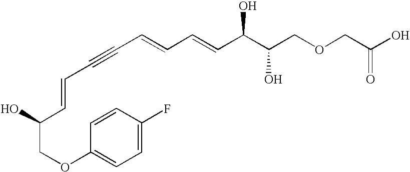
and forms a hydrate which exhibits characteristic peaks at d=9.8 Å and at d=4.6 Å.

11. A pharmaceutical composition comprising one or more pharmaceutically acceptable excipients and a therapeutically effective amount of a crystalline acid of claim 1.

12. A method of treating a disease-state characterized by inflammation in a mammal, the method comprising administering to the mammal in need thereof a therapeutically effective amount of a crystalline acid of claim 1.

13. The method according to claim 12 wherein the disease-state is an inflammatory or autoimmune disorder.

14. The method according to claim 12 wherein the disease-state is a pulmonary or respiratory tract inflammatory disorder.

15. A method of synthesizing a crystalline acid of Formula (II): 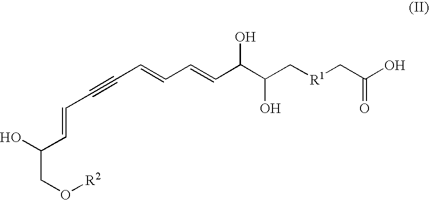
wherein:
R¹ is —O—, —S(O)ₜ— (where t is 0, 1 or 2), or a straight or branched alkylene chain; and
R² is aryl (optionally substituted by one or more substituents selected from alkyl, alkoxy, halo, haloalkyl and haloalkoxy) or aralkyl (optionally substituted by one or more substituents selected from alkyl, alkoxy, halo, haloalkyl and haloalkoxy);
as a single stereoisomer or any mixture of stereoisomers;
the method comprising:
1) mixing an alkali hydroxide base, in a suitable solvent, together with an ester of Formula (IV), in a suitable solvent: 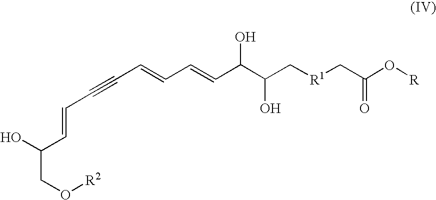
wherein R¹ and R² are as defined above, and R is alkyl or aryl;
2) acidifying the resulting mixture by treatment with an acid;
3) isolating the resulting crystals from the resulting suspension;
4) optionally washing the isolated crystals with a suitable solvent; and
5) drying the isolated crystals, to give the final product crystalline acid.

16. The method according to claim 15 wherein the suitable solvent for the alkali hydroxide base comprises an organic solvent and water.

17. The method according to claim 15 which comprises the additional step, prior to acidifying the mixture, of adding water in an amount sufficient to effect suitable crystallization of the product upon acidification

18. The method according to claim 15 wherein R¹ is —O— and R² is phenyl optionally substituted by one or more substituents selected from fluoro, chloro and iodo.

19. The method according to claim 15 wherein the final product crystalline acid is:
2-((2S,3R,4E,6E,10E,12S)-13-(4-fluorophenoxy)-2,3,12-trihydroxytrideca-4,6,10-trien-8-ynyloxy)acetic acid;
2-((2R,3R,4E,6E,10E,12S)-13-(4-fluorophenoxy)-2,3,12-trihydroxytrideca-4,6,10-trien-8-ynyloxy)acetic acid;
2-((2S,3S,4E,6E,10E,12S)-13-(4-fluorophenoxy)-2,3,12-trihydroxytrideca-4,6,10-trien-8-ynyloxy)acetic acid;
2-((2R,3S,4E,6E,10E,12S)-13-(4-fluorophenoxy)-2,3,12-trihydroxytrideca-4,6,10-trien-8-ynyloxy)acetic acid;
2-((2S,3R,4E,6E,10E,12R)-13-(4-fluorophenoxy)-2,3,12-trihydroxytrideca-4,6,10-trien-8-ynyloxy)acetic acid;
2-((2R,3R,4E,6E,10E,12R)-13-(4-fluorophenoxy)-2,3,12-trihydroxytrideca-4,6,10-trien-8-ynyloxy)acetic acid;
potassium 2-((2S,3S,4E,6E,10E,12R)-13-(4-fluorophenoxy)-2,3,12-trihydroxytrideca-4,6,10-trien-8-ynyloxy)acetic acid; or
potassium 2-((2R,3S,4E,6E,10E,12R)-13-(4-fluorophenoxy)-2,3,12-trihydroxytrideca-4,6,10-trien-8-ynyloxy)acetic acid.

20. The method according to claim 15, wherein the final product crystalline acid is 2-((2S,3R,4E,6E,10E,12S)-13-(4-fluorophenoxy)-2,3,12-trihydroxytrideca-4,6,10-trien-8-ynyloxy)acetic acid: [chemical structure]
